(19) United States
(12) Patent Application Publication
(10) Pub. No.: US 2008/0156646 A1
(43) Pub. Date: Jul. 3, 2008

(54) Nanostructured electrochemical biosensor with aptamer as molecular recognition probe

(76) Inventors: Nianqiang Wu, Morgantown, WV (US); William Phillip Petros, Morgantown, WV (US); Daniel Charles Flynn, Morgantown, WV (US); Peter Mico Gannett, Morgantown, WV (US)

Correspondence Address: WEST VIRGINIA UNIVERSITY RESEARCH CORPORATION, 886 CHESTNUT RIDGE ROAD, P.O. BOX 6224, MORGANTOWN, WV, 26506-6224, US

(21) Appl. No.: 11/986,884

(22) Filed: Nov. 27, 2007

Related U.S. Application Data
(60) Provisional application No. 60/875,263, filed on Dec. 15, 2006.

(51) Int. Cl. C12Q 1/25

(52) U.S. Cl. 204/450; 204/403.14

(57) ABSTRACT

The present invention details a nanostructured electrochemical biosensor based on aptamer as the molecular recognition probe. The biosensor is comprised of an electrochemical cell that can be plugged into an electric controller. This electrochemical biosensor contains a working electrode made of the micro-/nano-scale gold dot array pattern, on which aptamer is immobilized on the surface of the dot array as the molecular recognition probe. The aptamer is labeled with an electrochemical indicator. Reversible binding of an analyte to the aptamer causes the change in the conformation of aptamer, consequently brings the electrochemical indicator close to the electrode surface. This results in the electron transfer from the electrochemical indicator to the electrode, which can be read as a change in output current or potential. The invented biosensor is a portable point-of-care device that has higher sensitivity, allows the measurement of the analyte more rapidly and requires much less sample volume than is presently available with current methods of detection.

### CROSS-REFERENCE TO RELATED APPLICATIONS

This application claims priority of U.S. provisional application No. 60/875,263.

### REFERENCE TO SEQUENCE LISTING, A TABLE, OR A COMPUTER PROGRAM LISTING COMPACT DISC APPENDIX

Organization Applicant

Street:

City:

State:

Country:

PostalCode:

PhoneNumber:

FaxNumber:

EmailAddress

### <110> OrganizationName: West Virginia University

### Organization Applicant

Street: 886 Chestnut Ridge Road

City: Morgantown

State: WV

Country: USA

PostalCode: 25606

PhoneNumber:

FaxNumber:

EmailAddress:

### <110> OrganizationName: West Virginia University

### Application Project

<120> Title: Nanostructured Electrochemical Biosensor

<130> AppFileReference: 373

<140> CurrentAppNumber:

<141> CurrentFilingDate:

### Sequence

<213> OrganismName: Artificial

<400> PreSequenceString:

tttttgttt tgtaaaaa

<212> Type: DNA/RNA

<211> Length: 18

SequenceName: Seq 1

SequenceDescription:

### BACKGROUND OF THE INVENTION

The current method for monitoring cisplatin therapy is based on periodic blood analysis. The detection of cisplatin is currently based on large scale analytical instruments such as an Atomic Absorption Spectrometer (AAS) or a High Performance Liquid Chromatography (HPLC). AAS is the most common method used to measure platinum concentration. While the method is sensitive, it has several problems in that it needs immediate sample processing to avoid in vitro protein binding, the samples must be ultra-filtrated to remove plasma proteins, and the necessary instrumentation must be available. However, even after such processing the sample contains amino acids, glutathione, etc. The current existing technique for cisplatin detection is arduous and slow, being hampered by extensive sample pre-treatment, reagent preparation, and the need for immediate sample processing. In addition, the AAS is a large scale analytical instrument. Hence, neither can it be used as a point-of-care device, nor can it be potentially implanted for in-vivo detection.

### BRIEF SUMMARY OF THE INVENTION

The present invention can be embodied as a device for the rapid detection of the analyte in serum or other relevant samples to allow a physician to rapidly determine if the patient has the desired level of the analyte in their system.

Another aspect of the present invention is that it can be used as a point-of-care device or may, potentially, be implanted into a subject to determine if a combination of drug therapies promote or inhibit the desired biomarker.

The present invention can be further embodied as a nanostructured electrochemical biosensor. The biosensor is comprised of an electrochemical cell that can be plugged into an electric controller. The electrochemical cell contains a working electrode made of micro-/nano-patterns of a gold dot array that offers a fast response, high sensitivity, and a high signal-to-noise ratio. The electrochemical cell has a molecular recognition probe which is immobilized on the gold dots of the working electrode surface and confers analyte selectivity and specificity.

A further embodiment of the present invention can be the aptamer by artificial design, a single stranded DNA or RNA based molecular recognition probe that will reversibly bind desired analyte molecules, causing a conformational change in the molecular recognition probe, and thereby changing the current density to the electrode.

Another aspect of the present invention can be reusability resulting from the reversible binding of the analyte to the molecular recognition probe due to the molecular design of the molecular recognition probe.

A further aspect of the present invention is the reversible binding of the analyte cisplatin to detect the levels of cisplatin and platinum in the serum.

Another aspect of the present invention is the ability to make a molecular recognition probe for cisplatin that is ssDNA unless bound to cisplatin where the ssDNA undergoes a conformational change to allow for an electrochemical indicator to be moved close to the gold dot array allowing for an electrical output current or potential.

### BRIEF DESCRIPTION OF THE SEVERAL VIEWS OF THE DRAWING

FIG. 1 is a view of the electrochemical cell.

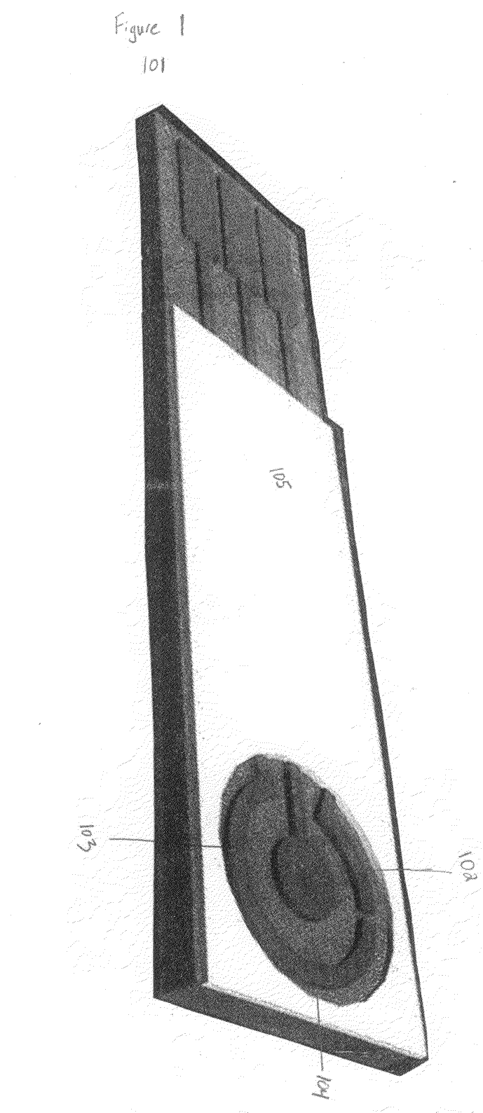

FIG. 2 is a view of the gold dot array pattern as the ultrafine working electrode.

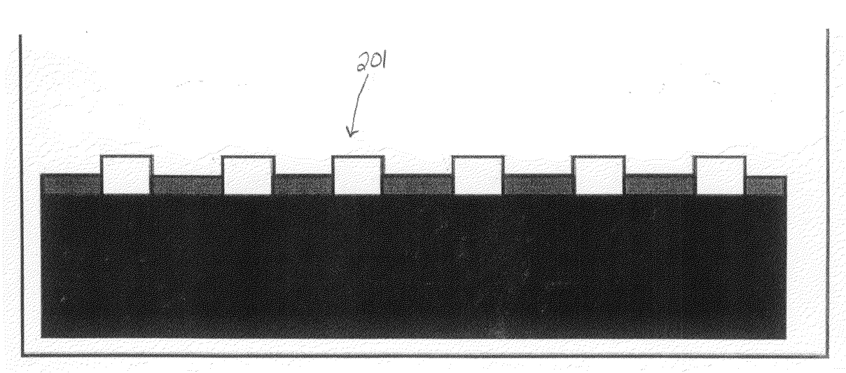

FIG. 3 is a view of the conformational change the molecular recognition probe undergoes during binding with cisplatin.

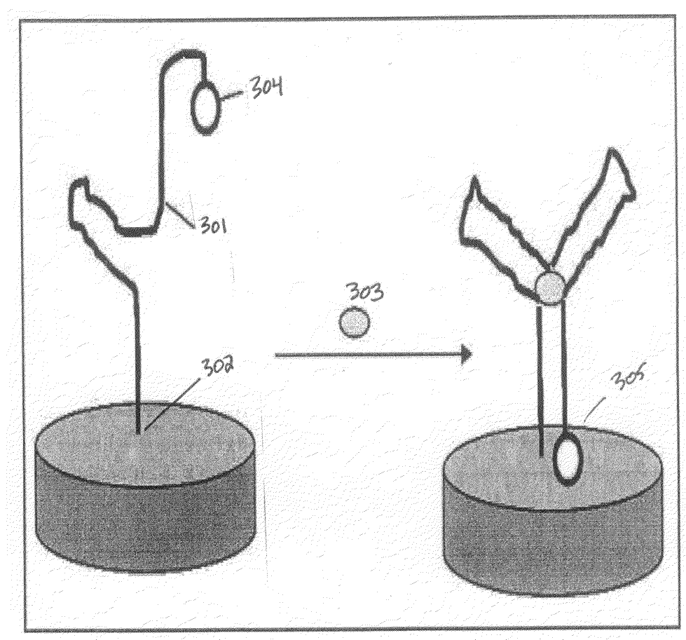

### DETAILED DESCRIPTION OF THE INVENTION

Electrochemical biosensors are increasingly finding applications in monitoring various analytes by utilizing drug-DNA interaction because they are relatively simple and inexpensive to build, reliable, and minimize the sample volume required. Moreover, electrochemical sensors can be fabricated in small chips which can be potentially implanted into the site of tumor removal. However, it remains a challenge to improve the sensitivity and the response time of the electrochemical biosensors. The present invention applies nanotechnology to biosensor fabrication and has opened a new pathway to improve the performance of biosensors. This invention leads to miniaturized of devices with high sensitivity and rapid response for real-time analysis of small volume samples.

The present invention is a nanostructured electrochemical biosensor. The biosensor comprises an electrochemical cell 101 that can be plugged into a portable electric controller. The electrochemical cell includes a reference electrode 102, a counter electrode 103 and the working electrode 104. In this three-electrode cell, the reference electrode is any electrode one skilled in the art would create such as an electrode made of Ag/AgCl. It has a stable and well-known electrode potential, which is used to measure electrochemical potential. The carbon counter electrode, also called an auxiliary electrode, is used only to make a connection to the electrolyte so that a current can be applied to the working electrode. The working electrode is made of micro-/nano-patterns of gold dot 201 array that offer fast response and high sensitivity with a high signal-to-noise ratio. All three electrodes used in an embodiment are connected to the electrical contacts on a plastic or other non-conductive substrate such as poly vinyl chloride (PVC) or poly vinyl chloride (PVC). 105. A molecular recognition probe 301, for example, a molecular recognition probe that specifically binds to cisplatin, is immobilized on the gold-dot array working electrode surface 302. This molecular recognition probe is immersed into a phosphate buffer solution or serum solution that contains the analyte 303, and a signal is generated based upon the reversible binding of the analyte (target molecules) to the molecular recognition probe.

Unique to this invention is that the molecular recognition probe links to an electrochemical indicator 304. Therefore, the molecular recognition probe can be any biomolecule that can alter its conformation and consequently alters the distance 305 separating the electrochemical indicator and the electrode surface. For example, MRPs such as aptamers, which are nucleic acid species, are good candidates, because they change conformation when they bind an analyte molecule. Molecular recognition probes can be designed from first principles, evolutionarily engineered through in vitro selection or, equivalently, by the SELEX (systematic evolution of ligands by exponential enrichment) method to specifically bind to various molecular analytes (targets) such as small molecules, proteins, nucleic acids, and even cells, tissues and organisms. This design is a general approach, which allows the biosensor to detect a broad range of analytes. Aptamers are excellent bio-recognition probes due to their unique chemical characteristics. As compared with other molecular recognition probes such as antibodies and enzymes, aptamers possess significant advantages including their small size, chemical simplicity, and flexibility. In addition, aptamers can be easily modified to incorporate electrochemical redox indicators, to allow immobilization, and can be reversibly denatured, conferring device reusability. An aptamer sensor can be operated in a wide variety of sample matrixes including non-physiological buffers and temperature conditions that would denature typical antibody formulations.

An electrical potential change is generated by a conformational change of the aptamer which, in turn, alters the separation between the redox active indicator and the working electrode when the analyte is present in solution. Methylene blue is often used as an electrochemical redox indicator (other redox indicators such as Phenanthroline Fe(II) are alternative options). This will lead to enhancement of electron transfer from the electrochemical indicator to the electrode, which can be read out in the electric controller as a change in output current or potential. The change in the sensing signal corresponds to the variation of the concentration of the analyte. The electric controller can read the electrochemical cell and display the results digitally and could be a standard device such as a CPU or palm sized digital display device or could be created by one skilled in the art specifically for the reading of the biosensor.

The result of the present invention is a point-of-care device that can potentially be implanted for in vivo detection. The present invention has a nanostructure in the working electrode which significantly improves the time response and the signal-to-noise ratio of the device. By varying the molecular recognition aptamer that is immobilized on the electrode the device can be used to detect a broad range of analytes.

One such analyte is cisplatin, a platinum based anti-cancer drug used to treat various types of cancers. In this embodiment of the present invention, an apatmer labeled with a redox indicator such as methylene blue is immobilized on the micro/nano-patterns and incubated with serum. Generation of a signal is based on the reversible binding of cisplatin to the single-stranded aptamer-redox indicator. If cisplatin is present, it binds to the aptamer which changes conformation so that the redox indicator is brought closer to the electrode surface. This leads to the enhancement of the electron transfer from methylene blue to the electrode and can be read out as a change in the output current or potential with the sensing signal corresponding to the concentration of the cisplatin.

The key to the aptamer sequence is the binding element present in the aptamer which contains two guanines arranged in such a way that when cisplatin binds to them the single strand DNA will form a hairpin and thereby locate the 3′-redox indicator near the gold surface as required such that the current flow will be altered. In addition, there is a portion of the sequence that will form a hairpin in the binding process, but without cisplatin will exist as single stranded DNA at body temperature. The sequence TTT-TTT-GTTT-TGT-AAA-AA is an example of such a sequence that meets the prescribed conditions. This molecular recognition probe could be made in any conventional way by one skilled in the art.

The preparation of the aptamer as a molecular recognition probe could be performed by a method that uses a CPG (Controlled Pore Glass) resin with a protected amine in a form suitable for preparation by automated DNA synthesis to which are chemically attached each DNA or RNA base of the aptamer and then terminating with a protected thiol. Other end groups could substitute the protected amino and thiol groups terminating the aptamer for groups that are used for attachment of the redox active group, such as methylene blue, and for attachment to the gold electrode, respectively. The aptamer could also contain non-natural DNA or RNA bases or other elements capable of mutual and reversible binding. The resulting aptamer is then deprotected with concentrated ammonia, followed by trichloroacetic acid, and finally treated with methylene blue, utilizing methods common to the art of aptamer preparation, to complete the construction of the aptamer. Attachment of the aptamer-redox indicator probe first requires cleaning the gold working electrode with piranha acid for thirty minutes followed by washing, in succession, with water, ethanol, and hexane, and drying under argon. The cleaned working electrode is then exposed to a solution of the aptamer-redox indicator probe in water, at a concentration typical of that used in the art, overnight, and then washed free of any unattached aptamer-redox indicator probe with water.

The nanostructured electrochemical biosensor could be further modified as an implantable chip. To ensure the passage of small molecules such as cisplatin and to serve as a barrier to biological species that might bind to the molecular recognition probe or degrade it, a membrane with a pore size of about 1000 MW (molecular weight) will be placed across the inlet. The membrane can be made from any of the materials typically used including but not limited to cellulose acetate, polysulfonate, or polyamide which have been cross-linked to such an extent that the desired pore size is achieved.

These terms and specifications, including the examples, serve to describe the invention by example and not to limit the invention. It is expected that others will perceive differences, which, while differing from the forgoing, do not depart from the scope of the invention herein described and claimed. In particular, any of the function elements described herein may be replaced by any other known element having an equivalent function.

What is claimed is:

1. An electrochemical cell comprising a non-conductive substrate, a plurality of electrodes connected to said substrate wherein said electrodes are also connected to electrical contacts and at least one of said electrodes is a working electrode wherein said working electrode is a gold dot micro/-nano patterned array with a molecular recognition probe wherein said molecular recognition probe contains an electrochemical redox indicator resulting in said molecular recognition probe binding to an analyte and said molecular recognition probe undergoing a conformational change causing said electrochemical indicator to alter the distance from said gold dot array surface.

2. The electrochemical cell of claim 1 wherein said plurality of electrodes are one or more of a reference electrode, a counter electrode, and a working electrode.

3. The electrochemical cell of claim 2 wherein said reference electrode is Ag/AgCl based.

4. The electrochemical cell of claim 2 wherein said counter electrode is carbon based.

5. The electrochemical cell of claim 1 wherein said non-conductive service is plastic based.

6. The electrochemical cell of claim 1 wherein said molecular recognition probe is an aptamer.

7. The electrochemical cell of claim 6 wherein said aptamer is a ssDNA or sRNA.

8. The electrochemical cell of claim 6 wherein said aptamer is [Seq1].

9. The electrochemical cell of claim 1 wherein said electrochemical redox indicator is methylene blue.

10. The electrochemical cell of claim 1 wherein said electrochemical redox indicator is phenanthroline Fe(II).

11. The electrochemical cell of claim 1 further comprising an electric controller capable of a digital display of a change in output current or potential when said electrochemical indicator changes distance from said gold dot array.

12. A method to reversibly bind cisplatin in a serum comprising exposing an electrochemical cell to a serum wherein said electrochemical cell contains a working electrode, a reference electrode and a counter electrode connected to a non-conductive substrate and electrical contacts, binding an the aptamer [Seq1] labeled with an electrochemical redox indicator wherein said aptamer is immobilized on a micro/nano patterned gold dot array working electrode surface of an electrochemical cell wherein cisplatin binds to said aptamer causing a conformational change in said aptamer causing a change in the current density to said electrode.

13. The method to reversibly bind cisplatin in a serum of claim 12 further comprising implanting said electrochemical cell into a subject.

14. A method of making an electrochemical cell comprising preparing an aptamer, adding a redox active group to said aptamer, attaching said aptamer to a gold dot micro/-nano patterned arrayworking electrode.

15. The method of making an electrochemical cell of claim 14 further comprising adding a membrane with a pore size of about 1000 MW across the inlet of said electrochemical cell.
